(19) United States
(12) Patent Application Publication
(10) Pub. No.: US 2002/0168376 A1
(43) Pub. Date: Nov. 14, 2002

(54) Compositions of multimeric profilin for diagnosis and treatment of allergies

(76) Inventor: Michael Babich, Auburn, CA (US)

Correspondence Address: BARNES & THORNBURG, 2600 CHASE PLAZA, 10 LASALLE STREET, CHICAGO, IL, 60603

(21) Appl. No.: 10/084,638

(22) Filed: Feb. 27, 2002

Related U.S. Application Data
(60) Provisional application No. 60/272,149, filed on Feb. 28, 2001.

(51) Int. Cl. A61K 39/00

(52) U.S. Cl. 424/185.1; 424/275.1

(57) ABSTRACT

Multimers of plant profilin or functionally equivalent peptides are a preferred form for diagnosis and treatment of allergies. Natural and synthetic whole molecules or fragments that represent the multimers are used 1) as hyposensitizing agents for treatment of allergies and 2) for screening patients to determine profilin allergenicity

This application claims priority from U.S. Provisional Application No. 60/272,149 filed Feb. 28, 2001.

### BACKGROUND OF THE INVENTION

Multimers of plant profilin are a preferred form for diagnosis and treatment of allergies. Natural and synthetic whole molecules or fragments that are functional equivalents of the multimers are used 1) as hyposensitizing agents for treatment for allergies; and 2) diagnostic agents for screening patients to determine profilin allergenicity.

Profilins are cytoskeletal proteins expressed in all eukaryotic cells that sequester G-actin and bind to membrane-associated phosphatidylinositol-4,5-bisphosphate (Carlsson et al., 1976; Theriot and Mitchison, 1993; Sohn and Goldschmidt-Clermont, 1994; Goldschmidt-Clermont and Janmey, 1991; Baalout, 1996; Lassing and Lindberg, 1985; Valenta et al.,1993), thereby affecting both cell morphology and signal transduction. Profilins have been identified and purified from multiple sources (e.g., human cells, tree, grass, weed pollens) and have been produced by recombinant DNA technology (Valenta et al., 1992a, b; Vrtala et al., 1996a, b; Susani, 1995; Pauli et al., 1996; Kwitakowski and Bruns, 1988; Honore et al., 1993).

The existence of human profilin multimers (i.e., profilin self-associations) was first reported by Babich et al (1996) in which tetramers (complexes of four profilin molecules interconnected; to form profilin4) were identified as the relevant high-affinity actin-binding form. Immunoblots, capillary zone electrophoresis and sodium dodecyl sulfate polyacrylamide gel electrophoresis (SDS-PAGE) analyses were interpreted to show that human profilin monomers of approximately 14.8 kDa molecular weight form multimers comprised mostly of dimers (profilin₂) and tetramers. Furthermore, functional significance was inferred by actin preferentially binding to the tetrameric form of profilin. Subsequently, another study showed that profilins from birch, human, and yeast self-associate (Mitterman, et al., 1998), but neither function nor clinical significance was addressed.

There is a wide range of homology (˜40-99%) among the various profilin monomers identified from humans, plants, and animals. Structural analysis has been reported that revealed remarkable homology between profiling from different plant species (Valenta et al., 1992; Vrlala et al., 1996). However, plant profilins appear to share common antibody recognition sites (epitopes); specifically, profilin-sensitive allergy patient IgE antibodies cross-react with different profilins. In addition, rabbit polyclonal antibodies raised against recombinant birch profilin (Valenta and Kraft, 1995) cross-react with virtually all plant profilins reported. The available data indicate that profilin from one plant source can cross-sensitize an individual to several plant species and may explain why some patients with Type I hypersensitivities display reactions to a wide range of distantly related pollens and foods.

An estimated 44 million patients (from North America, Europe, and Japan) suffer from Type I allergies to profilin which is found in plants, animals, and substances such as latex. Type I allergy symptoms include hay fever, runny nose, itching, wheezing and skin reactions, as well as the highly publicized fatal reactions to microscopic amounts of peanut. Type I allergies are also associated with the development of asthma. Thus, any aspects that are unique to profilin may, in turn, provide a basis for further study and development of allergy diagnosis and treatment.

European studies have reported that profilin isoforms isolated from various plant sources may act as generic-, or pan-allergens (Valenta et al., 1992a, b; 1991; Valenta and Kraft, 1995) and that approximately 20% of all pollen-allergic patients (with Type I allergies) display IgE reactivity to recombinant birch profilin (Valenta et al., 1992a, b; 1991; Valenta and Kraft 1995). A recombinant birch profilin, as well as natural profilins from birch, timothy grass and mugwort, are able to elicit IgE-mediated histamine release from basophils of pollen-allergic patients. A study of the North American population by the present inventor (Psaradellis et al., 2000) also demonstrated sensitivity of allergic patients to profilin. Thus, profilin may be a pan-allergen as well as being responsible for the sensitization and maintenance of a significant number of Type I allergic patients.

As discussed and cited in the work reporting discovery of human profilin multimeric forms (Babich, et al., 1996), investigators had not considered the existence of profilin multimers to have any relevance, even to the extent that they were often dismissed as extraneous proteins. Previous investigators were either unaware that plant profilin multimers existed, or did not address the issue of profilin multimers as an allergenic form. However, little is known regarding the biological importance of protein aggregation/self-association. The phenomenon is generally thought of as a biochemical attraction that takes place in which a biological role, if any, remains elusive. Even if a biological role is discovered, a clinical role does not necessarily become obvious. For example, in the allergy field, there are no specific multimeric -proteins being used to date for hyposensitization shots to set a precedent for the present application.

Overall, very few specific allergens have been identified; therefore identification of specific allergenic forms of profilin would be clinically useful. Consequently, few compositions are available for injection of a purified, specific causative allergy agent to induce hyposensitization. Most medication is directed towards treating allergy symptoms, but not the cause. Vaccination is the only treatment which is closest to curative; it is able to change the immune-system reaction pattern, stopping symptoms and, in certain patients, preventing the escalation of hay fever to asthma. Vaccines are used as hyposensitizing agents to convert the type of immunoglobulin/antibody response of the patient from IgE to predominantly Ig (also referred to as “sero-conversion”). IgE is the common response of the patients to clear their bodies of the allergen, but it also evokes side effects that are the commonly known allergy symptoms (e.g., runny nose, wheezing lungs, itchy eyes, skin rash, nausea), whereas IgG can help remove the allergen without such side effects. Successful hyposensitization vaccines thus render an IgG response that is minor compared to the elevated IgE levels against a given allergen.

Allergy vaccination treatments to date mostly consist of injection of a cocktail of extracts from allergenic substances, such as grass pollen, to which the patient is allergic. Cocktails are used because very few specific allergens are identified. By gradually increasing the dosage, the patient's immune response will change and the patient will eventually no longer show an allergic reaction to the allergens via sero-conversion. The availability of such compositions thereby improves treatment, but a more specific allergen (rather than cocktail) would be more effective, reproducibly prepared, and generally have less side effects.

### SUMMARY OF THE INVENTION

An aspect of the present invention is identification of profilin multimers as an allergenic form.

With regard to allergenicity, larger antigens such as multimers could present additional epitopes to elicit a greater IgE-mediated histamine release. This possibility was explored leading to the present invention, aspects of which include 1) that plant profilin forms multimers; and 2) multimeric forms are more allergenic than monomers. Recombinant plant profilin multimerization was studied and immunoassays were developed to assess IgE reactivity of individuals to plant profilin. The correlation between Type I hypersensitivities and reactivity to plant profilin within a population was examined in the U.S. (Illinois) and found to support the idea that profilin is a pan allergen in 20-30% of patients. Therefore, diagnostic and therapeutic uses of profilin multimers will have significant clinical impact.

The invention relates methods and compositions to hyposensitize a mammal. The compositions include production and/or purification of naturally occurring, synthetic, or recombinantly produced profilin (sources of monomers are listed in Table 3), which yields multimeric forms. The methods include the steps of:

(a) obtaining an immunogenic composition comprising multimeric profilin; and

(b) administering an effective dose of the composition successively in incremental doses until the mammal is hyposensitized.

The invention also relates utilizing multimeric profilin compositions for diagnostic means by immunoassays such as:

(a) administering an effective dose of compositions comprising multimeric profilin via established RAST (radio-allergosorbent test) or any skin tests known to those of skill in the art to diagnose patient sensitivity to profilin; and

(b) applying said composition in a tissue, blood, serum or plasma assay (e.g., enzyme-linked immunosorbant assay/ELISA; radioimmunoassay/RIA; immuno-radiometric assay/IRMA; RAST; luminescence immunoassay/LIA; magnetic allergoabsorbent test/MAT) to detect patient reactivity against profilin.

The profilin multimers may be in the form of a natural or synthetic peptide or polypeptide, or made by recombinant methods. The compositions may include pharmaceutically acceptable carriers or diluents known to those of skill in the art. Administration may be via parenteral, oral, nasal, inhalant or rectal routes. Treatment dosage is the amount which is sufficient to produce clinical effectiveness as measured by reduced IgE-related symptoms; diagnostic dosage is the amount which is sufficient to produce a measurable reaction in the respective procedure (e.g., skin tests=irritations; biological assay=detection of patient IgE binding to profilin).

Given that 1) 20-30% of Type I allergy patients have IgE that reacts with profilin; and 2) the present discovery of allergenic profilin multimers, then profilin multimers or congeners thereof (i.e., something closely resembling multimeric profilin or analogous to it) are important for diagnostic and vaccine treatment of these allergy types.

In agreement with profilin sizes determined from other sources (e.g., human, birch pollen) silver-strained SDS-PAGE gels and immunoblot analyses revealed that a significant 14.8 kDa protein was purified from Escherichia coli transformed with the cDNA of a plant (Zea mays) profilin isoform (ZmPRO1). Higher molecular weight proteins (particularly 60 kDa and 30 kDa) were also observed, which became predominant and larger (>90 kDa) in the absence of reducing agents. Human IgE reactivity to profilin was measured by enzyme linked immunosorbant assay (ELISA) that was developed using patient serum samples classified as either negative (no Type I allergies), positive (Type 1 plant allergies) or miscellaneous (i.e. allergies other than classical Type I plant allergies). The IgE-ZmPRO1 complexes were seen in three of nine patients with Type I plant allergies, compared with one of eight negative controls and three of 14 from the miscellaneous category. Dot filtration immunoblots were subsequently developed to absorb profilin diluted in the presence or absence of reducing agent to yield mostly monomeric or multimeric profilin, respectively. Immunoglobulin E from positive patients displayed a greater intensity of binding to ZmPRO1 under conditions that favored profilin multimers. In summary, recombinant ZmPRO1 profilin forms multimers and is suitable for a developed ELISA. Profilin has pan-allergenic potential, and profilin multimers have greater immunogenicity than monomers.

The combination of near-capacity protein loading and a relatively more sensitive SDS-PAGE staining procedure to identify the additional protein bands, compared with typical reports with Coomassie blue protein staining, may account for identifying multimer forms. In addition, plant and human profilin may be similar in their ability to resist chemical reduction. Computer-based molecular modeling of human profilin suggested a profilin-profilin interaction might occur that protects some of the disulfide bonds from harsh reducing agents (FIG. 5).

### BRIEF DESCRIPTION OF THE DRAWINGS

FIG. 1 shows a photograph of silver-stained SDS-PAGE separation of Escherichia coli transformed with Zea mays Zm PRO1 cDNA; the protein encoded by ZmPRO1 cDNA was purified by affinity bead slurry separation as described in the Materials and Methods; lane 1, non-transformed E. coli (negative control); lane 2, E. coli containing the pET23a/ZmPRO1 vector; lane 3, E. coli containing the pET23a/ZmPRO1 vector+IPTG to induce expression of the profilin protein; arrows show monomeric profilin; protein molecular weight marker migrations are listed on the right (in kDa).

FIG. 2 shows a photograph of an affinity column purified Zea mays Zm PROI from transformed Escherichia coli; (a) results are similar to silver-stained SDS-PAGE in FIG. 1, but profilin is separated by affinity column chromatography as described herein in the Materials and Methods; lane 1, under reducing conditions (+β-mercaptoethanol (BME); arrow depicts location of monomeric profilin; lane 2, under non-reducing conditions (−BME); (b) corresponding immunoblot of samples run in parallel with (a) lane 3, +BME; lane 4, −BME; rabbit anti-ZmPRO1 and horseradish peroxidase-conjugated secondary goat anti-rabbit IgG method was used to visualize immunologically distinct profilin proteins; protein molecular weight marker migrations are listed on the left (in kDa).

FIG. 3 shows a photograph of wells from an enzyme-linked immunosorbant assay developed for plant profilin; wells 1-2, using secondary antibody alone; wells 3-4, using primary antibody+secondary antibody; wells 5-6, using primary antibody+secondary antibody+Zea mays Zm PRO1 profilin; tris-buffered saline+ZmPRO1 profilin (i.e. negative control) gave no measurable optical density (not shown).

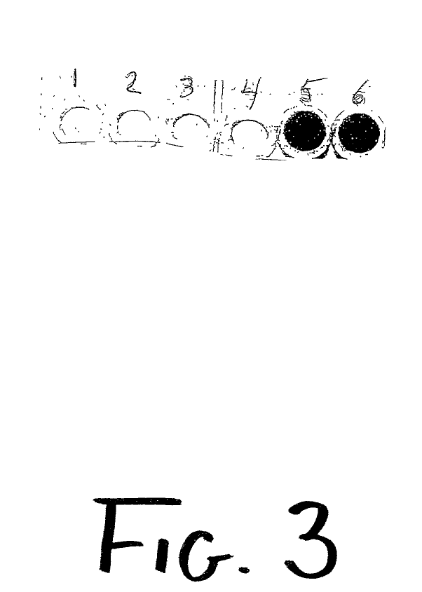

FIG. 4 shows a dot-filtration immunoblot of Zea mays ZmPRO1 profilin and human IgE. Profilin was adsorbed and filtered onto a dot-filtration apparatus under conditions that favor either monomers [+β-mercaptoethanol (BME)] or multimers (−BME), prior to addition of serum from patients declaring allergies [serum (+)] or without allergies [serum (−)]; control, rabbit anti-ZmPRO1 antibody (positive control); triplicate well determinations of the colorimetric assay are shown for all samples (background, −BME+profilin+secondary antibody+metal diaminobenzidine substrate; the background was no different when+BME was included); quantitative values (mean±SEM) for the intensity of darkness were calculated as described herein in the Material and Methods and are presented next to each row; student's t-test revealed significance levels of *P<0.05 or **P<0.01 for −BME versus corresponding +BME rows consisting of a protein with an amino acid sequence.

FIG. 5 shows computer-based molecular analysis of profilin self-association. The structure of crystalline profilin human profilin I (Metzler et al., 1995) was downloaded onto a computer for molecular modeling and analyses of the “best fit” to for dimerization. Software to analyze profilin structures included: QUANTA (the core program; Molecular modeling, graphics and manipulation for quality graphics and stereochemical insight), CHARMm (Chemistry at Harvard Molecular modeling; via the Karplus laboratory at Harvard, Mass.) for multiple physical chemistry manipulations (e.g., interactions, reactions, free energy calculations, energy minimization, etc.); UHBD (University of Houston Brownian Dynamics suite of programs from the McCammon laboratory) for Poisson-Boltzmann calculations, Brownian dynamics, pKₐ, enzyme-substrate interactions; UHBDINT to interface QUANTA with UHBD; QPROTN, for protein modeling system to predict 3-D structures from primary amino acid sequences using homology modeling; search and retrieve 3-D crystal structures of homologous sequences to the target sequence and construct models based on such structures. A high probability form of profilin self-association is shown in FIG. 5 Key:

Grey=represents structures of two human profilin I molecules that constitute the dimer, each containing the following:

White=actin binding domains

Black=cysteine residues with associated sulphur groups

Among the possible sulphydryl bonding between the three integral cysteines (amino acid postion numbers 16, 70, 127) numbers 16 and 127, each from a different protein moiety (the two mostly grey interconnected structures), were nearby and available in a conducive steric fit to form two disulfide bridges (S—S bonds) between the twin molecules (arrow pointing to the black interconnected molecules). Three dimensional conformation also revealed a relatively protective pocket surrounding the S—S bonds. A near 90 degree turn is observed between the two profilins with the actin binding domains and cysteine #70 accessible (e.g., can be seen as the black exposed molecule on the right profilin moiety). Unique regions that occur as a consequence of profilin-profilin binding (shown between the brackets) represent putative epitopes for allergic/IgE reactions, and therefore have amino acid sequences that can be used to develop novel peptides for the treatment and diagnosis of profilin-related type I allergies.

### DETAILED DESCRIPTION

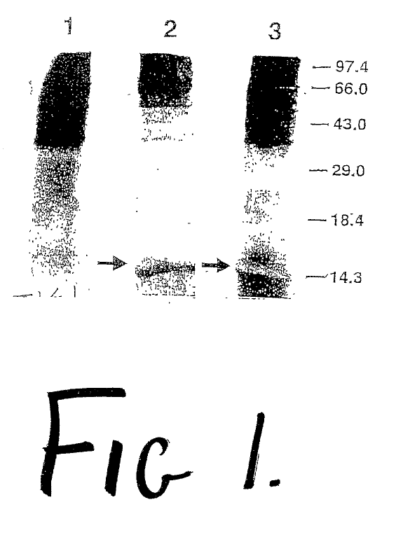

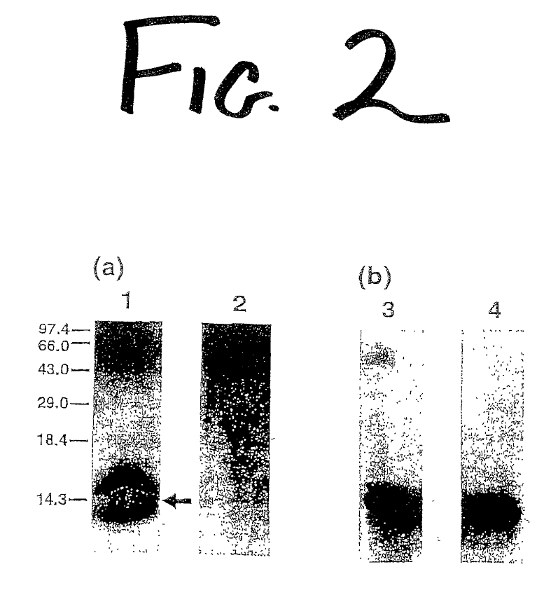

Allergy patients' IgE has been shown to cross react among profilins from different sources. The present invention is directed to profilins that are related to allergens which exist in a variety of plant species (trees, grass, weeds), foods such as peanut, and to the profilins found in humans. A novel aspect of the invention is that profilin multimers are the preferred, if not the exclusive, allergenic form. The size of the multimeric is greater than the monomeric form, including those wherein multiples of a monomer (approximately 14 kDa) could range in size at or between 28, 36, and 60 kDa, or those multimers above 100 kDa as described for the SDS-PAGE gels of FIGS. 1 and 2. The multimers may arise from self-association of profilin with the same identity (homomultimers), or may arise from cross-association between profilins (heteromultimers). An example of heteromultimers would be where the family of corn pollen profilins bind each other to form multimers; another example would be cross species heteromultimers (e.g., corn-birch pollen profilin complexes). Although heteromultimers have not been detected, conceivably a unique sequence may be identified from such heteromultimers that is ultimately the most potent and generically applicable form for treatment and/or diagnosis of profilin-sensitive type I allergies.

Profilin multimers may be made by methods known to those skilled in the art: e.g. by purification from their original sources in nature, by chemical synthesis, or recombinant DNA technology. The profilin or profilin peptides and fragments are subsequently purified by common affinity chromatography methods using poly-1-proline (Babich, et al., 1996; Janmey, 1991) or HPLC (high performance liquid chromatography). The term “synthetic” used herein includes all peptides and polypeptides produced by cloning and expression of the nucleotide sequences (Psaradellis, et al., 2000; Sambrook and Russell, 2001) or by commercially available chemical synthesis based upon the encoded nucleotide sequences, or a specifically designed amino acid sequence derived from known profilin amino acid sequences.

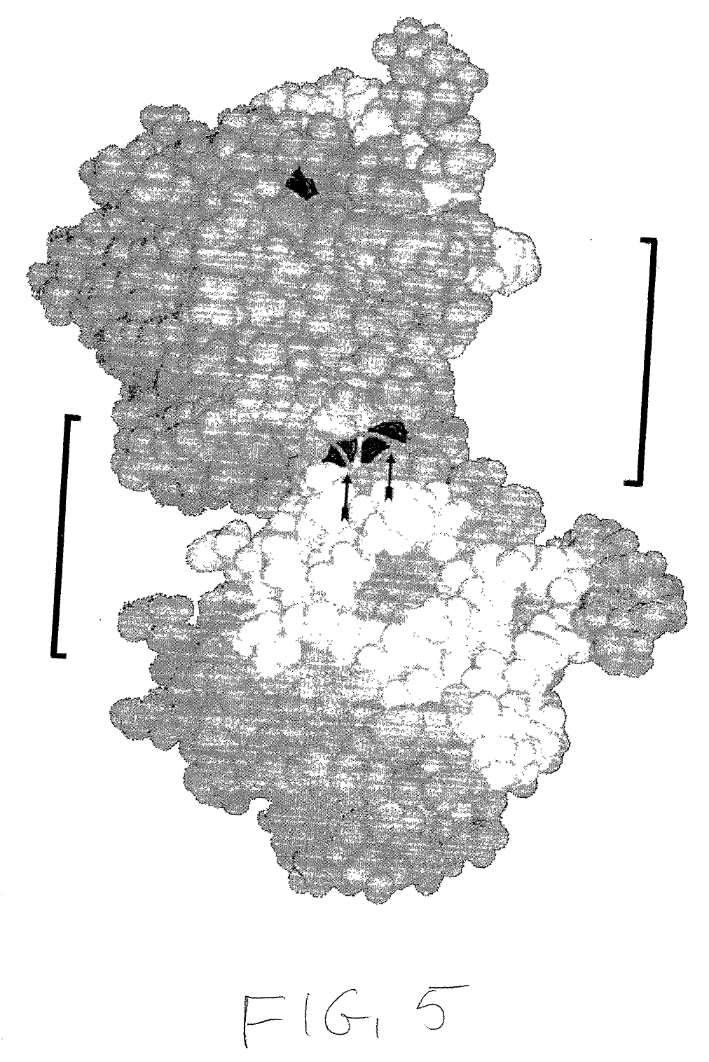

In addition to making whole profilin molecules that multimerize, synthetic peptides may also have the following requirements to make novel profilin-based peptides and polypeptides for therapy and diagnostics: 7-21 amino acids in length, contain at least one proline, and contain at least one acidic amino acid. The requirements are derived from a collective prediction from each of the following: 1) a minimum size that can be made efficiently, is sufficient for immune recognition, yet small enough to reduce potential side effects compared to larger molecules; 2) bends that occur within and among profilins upon multimerization can be mimicked in the peptides by proline; 3) charged amino acids for water solubility and potentially facilitate antibody-peptide interactions; 4) a plausible multimeric structure for profilin multimers was deduced from computer modeling (FIG. 5) which is consistent with a role for prolines and charged residues on the outside. Therefore, a person with IgE most likely would produce antibodies against the exposed portion of profilin complexes that result from multimerization or to a portion of the amino acid sequence from one profilin molecule that continues the sequence from the adjoining additional profilin molecule shown in the FIG. 5.

Table 3 shows the sequences of profilins with allergic potential. The sequences form the basis to develop multimeric profilins and, given the previously described parameters, to make peptide fragments for allergy treatment and diagnoses.

The ability of plant profilin to form clinically relevant multimers from human and a variety of plant species is a novel aspect of the present invention. The biochemical data and computer-based modeling were in agreement that profilin from various species can form multimers. Furthermore, the data from FIG. 5 show that profilin forms multimers that remain strongly attached due to strong chemical bonds (sulphydryl bonds) that are relatively protected from harsh reducing agents (which normally break such bonds), and the chemical free energy (favorable state) for two profilin molecules is to self-associate. Thus, the nature for two profilin molecules is to self-associate, which would explain why profilin multimers exist along with monomers.

As discussed under Background of the Invention, multimers were previously either unobserved, dismissed as contaminants, or not studied for clinical relevance. Two other reasons become apparent from the results (1) the lack of rabbit anti-plant profilin IgG to recognize human profilin (which forms multimers) could be construed as evidence that plant multimers do not exist because if the antibodies recognized human profilin multimers then it could have pointed investigators to look for plant profilin multimers; and (2) a more sensitive stain was used to detect proteins in the present SDS-PAGE experiments. For example, Coomassie blue was a preferred stain used by others to detect proteins with minimal background, thereby rendering a “cleaner” gel and preferentially detecting the most abundant proteins loaded onto the gel. In contrast, silver staining used herein is more sensitive and picks up background proteins. The gels are rather dark and protein bands are sometimes blended in with many other proteins that are close in size, in addition to producing a darker background (e.g., FIGS. 1 and 2). Thus, much of the published work in this area shows Coomassie blue stained gels, which could explain, in part, why the multimers were previously not evident or noted.

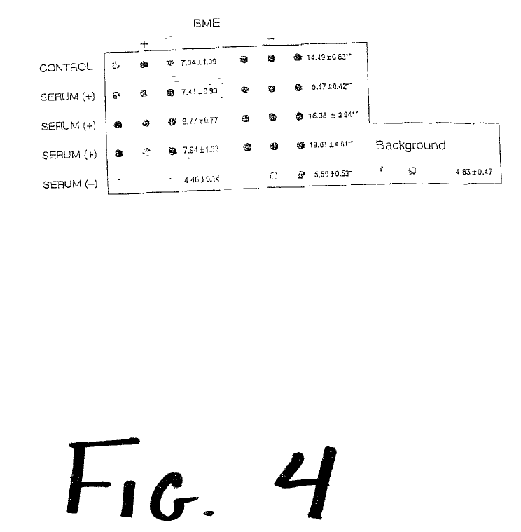

Indeed, established methods of profilin isolation have often yielded extraneous and unidentified proteins (discussed in Babich, et al., 1996) that are ≧2 times the recognized size of the 12-15 kDa cytoskeletal molecule. These bands were dismissed as contaminations, rather than sought as dimers or other profilin multimers. Furthermore, profilin used in allergy studies was purified or synthesized without determining the composition (mono-multimer) when testing for allergenicity. For instance, other investigators only focused on an assumed profilin monomer as a product for the diagnosis and therapy of allergic diseases to the extent that the very size of profilin monomers (14 kDa) has been used to name (P14) the patented product (Valenta, et al., 1996; U.S. Pat. No. 5,583,046 and U.S. Pat. No. 5,648,242). In contrast, as discussed in Psaradellis et al, 2000 and shown in FIG. 4, profilin multimers are proposed as the preferred, if not exclusive, allergenic form.

Clinical relevance for profilin multimers was obtained by an assay that demonstrated preferential binding of IgE from humans with Type I allergies. Larger profilin multimers were more allergenic due to their size and novel antigenic presentation to a susceptible human, thereby inducing an allergic reaction mediated by IgE. Use of this multimeric property of profilin includes: 1) the treatment of allergies; and 2) in diagnostic methods known to those of skill in the art (e.g., ELISA, RIA, IRMA, RAST, LIA, MAT) to determine patient allergic reactivity. Profilin to be used in these assays includes whole multimer profilin molecules (natural or synthetic), peptide fragments (natural or synthetic), or peptide fragments (synthetic neo-antigens) derived from the profilin structure that are either exposed for reaction upon multimerization or appear uniquely through profilin-profilin interactions. The proteins and peptides are either: a) acquired from plants or human tissues by standard purification methods (e.g., poly-l-proline affinity column purified profilin shown in FIGS. 1 and 2; or b) made by those skilled in the art using standard commercially available chemical synthesis; or c) recombinant DNA technology.

Profilin was purified from various natural sources using poly(1-proline)-sepharose 4B affinity chromatography as previously described (Babich, et. al., 1996; Psaradellis, et. al., 2000; Janmey, 1991). Whether isolated directly from cells of interest, or synthesized, the profilin was purified away from all other constituents by pouring onto a 10 ml poly(l-proline)-sepharose 4B column. Actin and profilin eluted with 4M and 8M urea, respectively, were concentrated by centrifugation (centriprep-3, Amicon Inc., Beverly, MA). Profilin was initially washed in G-buffer (0.1 mM CaCl₂, 0.2 mM ATP, 0.5 mM DTT, 2 mM Tris-HCl, pH 7.2), concentrated (1-3 mg/ml) and stored in 2 mM Tris-HCl (pH 7.4)/0.1 mM CaCl₂ at −20° C. until use. The source of profilin may be through recombinant DNA technology (Psaradellis, et al., 2000; Sambrook and Russell, 2001), such as for corn pollen profilin ZmPRO1 described further under Materials and Methods (Expression and purification of ZMPRO1 profilin).

Screening patients for specific allergens is of diagnostic use in a clinical setting. The proteins of the present invention are used to identify allergy patients that are sensitive to profilin and are used as hyposensitizing agents for patient sero-conversion. If a patient has IgE that recognizes profilin, then a hyposensitization reagent (i.e. “allergy shot”) is developed from the present discovery whereby the IgG becomes the patient's primary immune response to produce clinical benefits (i.e., clear the body of allergen without side effects associated with IgE).

The ELISA developed was subsequently used to measure Type 1 allergy patient IgE recognition of ZMPRO1 profilin. There was one positive reactivity to profilin among patients with no known allergies (one of eight); minimal reactivity in those with miscellaneous (e.g., penicillin, fabric, dust) non-type-I allergies (three of 14) and significant reactivity among those declaring type I allergies to pollen (three of six). The raw data from the three samples that gave a positive reaction to profilin (Table 2) showed a strong IgE reactivity to profilin with minimal background (i.e. a relatively high ratio of the optical densities when serum was added to profilin-coated wells vs. non-profilin- coated wells). The results agree with previous work (Valenta, et al., 1992; 1991, and 1995; Pauli 1996) that Type I allergy patients are immunoreactive to plant profilin and indicate that the current approach will be useful to screen for patients with type I allergies.

The allergenic potential of profilin monomers and multimers was tested by dot-filtration immunoblot analysis (FIG. 4). Although the ELISA is more sensitive and quantitative, the presence of reducing agents used to favor a monomeric profilin state (due to breaking of the sulphydryl inter-profilin bonds, but with incomplete effects as addressed earlier) would hinder the adsorption of profilin to plastic wells. Thus, a dot blot filtration apparatus was used to remove the reducing agents. In all instances, a greater response was measured from IgE recognition of +BME/profilin (i.e. profilin as a predominantly multimeric form) compared with−BME/profilin. In contrast to the relatively weak signal from negative control patients (i.e. serum(−), IgE From the positive serum category displayed a significantly greater recognition of profilin (regardless of ±BME), thereby implicating higher profilin orders as allergens.

Among the nine patients who declared Type 1 allergies to plant pollens, three showed significant reactivity to ZmPRO1 profilin with the developed ELISA. Three additional serum samples from the miscellaneous category (e.g. dust) also yielded a positive response. This might be expected, considering that many Type 1 allergy patients often include dust as an allergen; furthermore, the identification of profilin as an IgE-binding component in latex (Vallier et al., 1995) raises the issue that allergens considered outside the conventional spectrum of type 1 candidates may indeed be recognized by Type 1 allergy patient IgE.

The relatively greater recognition of ZmPRO 1 profilin multimers by IgE reveals novel aspects of plant profilin as a proposed pan-allergen. Greater recognition of profilin multimers is not due to simple additive effects, because the same amount of total profilin was added to each well used in the dot-immunoblot experiments. Thus, it appears that profilin multimers act in synergy to either sterically facilitate access to binding sites or to present unique epitopes. It is likely that: (i) more Type 1 allergy patients than previously estimated have IgE that recognize profilin; and (ii) profilin multimers are causative agents for Type 1 allergies.

TABLE 1
Enzyme-linked immunosorbant assay for human IgE reactivity
with ZmPRO1 profilin

| Category | + Reaction | − Reaction |
|---|---|---|
| Negative | 1 | 7 |
| Miscellaneous | 3 | 11 |
| Positive | 3 | 6 |

Positive (+) or negative (−) reaction of profilin with serum samples from the three patient categories (N=30 samples; quadruplicate determinations for each point), Zm=Zea mays.

TABLE 2
Raw data from the positive samples

| | OD (x10¹³) of + | |
|---|---|---|
| Patient sample no. | profiling coated wells | Declared Allergies |
| 25 | 940/80 | Hay Fever |
| 74 | 630/340 | Trees, pollen |
| 90 | 214/12 | Trees, grasses, strawberries |

The ratio of optical densities (OD) from profilin-coated versus non-coated wells for the three positive samples identified in Table 1 (mean OD shown; standard errors were within 10% of the mean).

TABLE 3

| Protein Isoform | Accession | Sequence |
|---|---|---|
| Api g4 (celery) | Q9XF37 | 1 mswqayvddh lmcevegnpg qtltaaaiig hdgsvwaqss |
| | | tfpqikpeei agimkdfdep |
| | | 61 ghlaptglyl ggakymviqg epnavirgkk gsggvtikkt |
| | | gqalvfgvyd epvtpgqcnv |
| | | 121 iverlgdyli dqgl |
| Ara h5 (peanut) | Q9SQI9 | 1 mswqtyvdnh llceiegdhl ssaailgqdg gvwaqsshfp |
| | | qfkpeeitai mndfaepgsl |
| | | 61 aptglylggt kymviqgepg aiipgkkgpg gvtiektnqa |
| | | liigiydkpm tpgqcnmive |
| | | 121 rlgdylidtg l |
| Bet v2 (birch tree pollen) | P25816 | 1 mswqtyvdeh lmcdidgqas nslasaivgh dgsvwaqsss |
| | | fpqfkpqeit gimkdfeepg |
| | | 61 hlaptglhlg gikymviqge agavirgkkg sggitikktg |
| | | qalvfgiyee pvtpgqcnmv |
| | | 121 verlgdylid qgl |
| Cyn d12 (bermuda grass) | O04725 | 1 mswqayvddh lmceieghhl tsaaiighdg tvwaqsaafp |
| | | afkpeemani mkdfdepgfl |
| | | 61 aptglflgpt kymiviqgepg avirgkkgsg gvtvkktgqa |
| | | lvigiydepm tpgqcnmvie |
| | | 121 klgdylieqg m |
| Gly m3 (soybean) GmPROI | O65809 | 1 mswqayvddh llcdiegnhl thaaiigqdg svwaqstdfp |
| | | qfkpeeitai mndfnepgsl |
| | | 61 aptglylggt kymviqgepg avirgkkgpg gvtvkktgaa |
| | | liigiydepm tpgqcnmvve |
| | | 121 rpgdylidqg y |
| Gly m3 (soybean) GmPROII | O65810 | 1 mswqayvddh llcgiegnhl thaaiigqdg svwlqstdfp |
| | | qfkpeeitai mndfnepgsl |
| | | 61 aptglylggt kymviqgepg avirgkkgpg gvtvkktgaa |
| | | liigiydepm tpgqcnmvve |
| | | 121 rlgdylidqg y |
| Hel A2 (Sunflower) | O81982 | 1 mswqayvdeh lmcdiegtgq hltsaailgl dgtvwaqsak |
| | | fpqfkpeemk giikefdeag |
| | | 61 tlaptgmfia gakymvlqge pgavirgkkg aggicikktg |
| | | qamimgiyde pvapgqcnmv |
| | | 121 verlgdylle qgm |
| Hev b8 (latex) | CAB51914 | 1 mswqayvddh lmceiegnhl saaaiigqdg svwaqsanfp |
| | | qfkseeitgi msdfhepgtl |
| | | 61 aptglyiggt kymviqgepg avirgkkgpg gvtvkktnqa |
| | | liigiydepm tpgqcnmive |
| | | 121 rlgdylidqg y |
| Hev b8 (latex) Profilin I | O65812 | 1 mswqtyvder lmceiegnhl taaaiigqdg svwaqssnfp |
| | | qfkseeitai msdfdepgtl |
| | | 61 aptglhlggt kymviqgeag avirgkkgpg gvtvrktnqa |
| | | liigiydepm tpgqcnmive |
| | | 121 rlgdylleqg m |
| Hev b8 (latex) Profilin II | Q9STB6 | 1 mswqayvddh lmceiegnhl saaaiigqdg svwaqsanfp |
| | | qfkseeitgi msdfhepgtl |
| | | 61 aptglyiggt kymviqgepg avirgkkgpg gvtvkktnqa |
| | | liigiydepm tpgqcnmive |
| | | 121 rlgdylidqg y |
| Hev b8 (latex) Profilin III | Q9M7N0 | 1 mswqtyvdeh lmcdidghhl taaaiighdg svwaqsssfp |
| | | qfkpeevaai mkdfdepgsl |
| | | 61 aptglhlggt kymviqgepg avirgkkgsg gitvkktgqa |
| | | liigiydepl tpgqcnmive |
| | | 121 rlgdylleqg m |
| Hev b8 (latex) Profilin IV | Q9M7M9 | 1 mswqtyvddh lmcdidghrl taaaiighdg svwaqsssfp |
| | | qfksdevaai mkdfdepgsl |
| | | 61 aptglhlgst kymviqgepg avirgkkgsg gitvkktsqa |
| | | liigiydepl tpgqcnmive |
| | | 121 rlgdylleqg m |
| Hev b8 (latex) Profilin V | Q9M7M8 | 1 mswqtyvddh lmcdidghrl taaaiighdg svwaqssgfp |
| | | qfksdevaav mkdfdepgsl |
| | | 61 aptglhlggt kymviqgepg avirgkkgsg gitvkktgqa |
| | | liigiydepl tpgqcnmive |
| | | 121 rlgdylleqg m |
| Hev b8 (latex) Profilin VI | Q9LEI8 | 1 mswqtyvddh lmcdidghrl taaaiighdg svwaqsssfp |
| | | qfksdevaav mkdfdepgsl |
| | | 61 aptglhlggt kymviqgepg avirgkkgsg gitvkktgqa |
| | | liigiydepl tpgqcnmive |
| | | 121 rlgdylldqg l |
| Mer a1 (Mercurialis Annua) | O49894 | 1 mswqtyvddh lmcdidgqgq hlaaasivgh dgsiwaqsas |
| | | fpqlkpeeit gimkdfdepg |
| | | 61 hlaptglyia gtkymviqge sgavirgkkg sggitikktg |
| | | qalvfgiyee pvtpgqcnmv |
| | | 121 verlgdylie qgm |
| Ole e2 (olive tree pollen) | P19963 | 1 mswqayvddh lmcdieghed hrltaaaivg |
| Profilin I | | hdgsvwaqsa tfpqfkpeem ngimtdfnep |
| | | 61 ghlaptglhl ggtkymviqg eagavirgkk gsggitikkt |
| | | gqalvfgiye epvtpgqcnm |
| | | 121 vverlgdylv eqgm |
| Ole e2 (olive tree pollen) | O24170 | 1 mswqayvddh lmcdiegheg hrltaaaivg |
| Profilin II | | hdgsvwaqsa tfpqfkpeem ngimtdfnep |
| | | 61 ghlaptglhl ggtkymviqg eagavirgkk gsggitikkt |
| | | gqalvfgiye epvtpgqcnm |
| | | 121 vverlgdyll eqgl |
| Ole e2 (olive tree pollen) | O24171 | 1 mswqayvddh lmcdiegheg hrltaaaivg |
| Profilin III | | hdgsvwaqsa tfpqfkpeem ngimtdfnep |
| | | 61 ghlaptglhl ggtkymviqg eagavirgkk gsggitikkt |
| | | gqalvfgiye epvtpgqcnm |
| | | 121 vaerlgdyll eqgl |
| Phl p11 (timothy grass) | P35079 | 1 mswqtyvdeh lmceieghhl asaailghdg tvwaqsadfp |
| Profilin I | | qfkpeeitgi mkdfdepghl |
| | | 61 aptgmfvaga kymviqgepg rvirgkkgag gitikktgqa |
| | | lvvgiydepm tpgqcnmvve |
| | | 121 rlgdylveqg m |
| Phl p11 (timothy grass) | O24650 | 1 mswqtyvdeh lmceieghhl asaailghdg tvwaqsadfp |
| Profilin II/IV | | qfkpeeitgi mkdfdepghl |
| | | 61 aptgmfvaga kymviqgepg avirgkkgag gitikktgqa |
| | | lvvgiydepm tpgqcnmvve |
| | | 121 rlgdylveqg m |
| Phl p11 (timothy grass) | O24282 | 1 mswqtyvdeh lmceieghhl asaaifghdg tvwaqsadfp |
| Profilin III | | qfkpeeitgi mkdldepghl |
| | | 61 aptgmfvaaa kymviqgepg avirgkkgag gitikktgqa |
| | | lvvgiydepm tpgqcnmvve |
| | | 121 rlgdylveqg m |
| Pru av4 (sweet cherry) | Q9XF39 | 1 mswqayvddh lmcdidgnrl taaailgqdg svwsqsatfp |
| | | afkpeeiaai lkdldqpgtl |
| | | 61 aptglflggt kymviqgeag avirgkkgsg gitvkktnqa |
| | | liigiydepl tpgqcnmive |
| | | 121 rlgdylieqg l |
| Pyr c4 (pear) | Q9XF38 | 1 mswqayvddh lmcdidghhl taaailghdg svwaqsstfp |
| | | kfkpeeitai mkdfdepgsl |
| | | 61 aptglhlggt kymviqgegg avirgkkgsg gvtvkktsqa |
| | | lvfgiyeepl tpgqcnmive |
| | | 121 rlgdylidqg l |
| Zm PRO1 (corn) ZmPRO I | P35081 | 1 |
| | | mswqtyvdehlmceieghhltsaaivghdgatwaqstafpefkpeema |
| | | aimkdfdepghl |
| | | 61 aptglilggtkymviqgepgavirgkkgsggitvkktgqs |
| | | liigiydepmtpgqcnlvve |
| | | 121 rlgdylleqgm |
| Zm PRO1 (corn) ZmPRO II | P35082 | 1 mswqayvdehlmceieghhlaaaaivghdgaawaqstafp |
| | | efktedmanimkdfdepghl |
| | | 61 |
| | | aptglflgptkymviqgepgavirgkkgsggitvkktgqalvvgiydepmtp |
| | | gqcnmvve |
| | | 121 rlgdylleqgm |
| Zm PRO1 (corn) ZmPRO III | P35083 | 1 mswqtyvdehlmceieghhlssaaivghdg avwaqstafp |
| | | qfkpeemtni ikdfdepgfl |
| | | 61 apiglflgptkymviqgepg |
| | | avirgkkgsggitvkktgqalvigiydepmtpgqcnmvve |
| | | 121 rlgdylveqgl |
| Zm PRO1 (corn) ZmPRO IV | O22655 | 1 mswqayvdehlmceiegqhlsaaaivghdgsvwaqsesfp |
| | | elkpeevagiikdfdepgtl |
| | | 61 aptglfvggtkymviqgepgvvirgkkgtggitikktgms |
| | | liigvydepmtpgqcnmvve |
| | | 121 rlgdylieqgf |
| Zm PRO1 (corn) ZmPRO V | Q9FR39 | 1 mswqayvddhllcdiegqhlsaaaivghdgsvwaqsenfp |
| | | elkpeevagmikdfdepgtl |
| | | 61 |
| | | aptglfvggtkymviqgepgvvirgkkgtggitikktgmsliigiydepmtpg |
| | | qcnmvve |
| | | 121 rlgdylieqgf |
| Human Profilin I | P07737 | 1 magwnayidn lmadgtcqda aivgykdsps vwaavpgktf |
| | | vnitpaevgv lvgkdrssfy |
| | | 61 vngltlggqk csvirdsllq dgefsmdlrt kstggaptfn |
| | | vtvtktdktl vllmgkegvh |
| | | 121 gglinkkcye mashlrrsqy |
| Human Profilin II Isoform a | NP_444252 | 1 magwqsyvdn lmcdgccqea aivgycdaky |
| | | vwaataggvf qsitpieidm ivgkdregff |
| | | 61 tngltlgakk csvirdslyv dgdctmdirt ksqggeptyn |
| | | vavgragrvl vfvmgkegvh |
| | | 121 ggglnkkays makylrdsgf |
| Human Profilin II Isoform b | NP_002619 | 1 magwqsyvdn lmcdgccqea aivgycdaky |
| | | vwaataggvf qsitpieidm ivgkdregff |
| | | 61 tngltlgakk csvirdslyv dgdctmdirt ksqggeptyn |
| | | vavgragral vivmgkegvh |
| | | 121 ggtlnkkaye lalylrrsdv |

### EXAMPLES

### Example 1

### Production and Purification of Multimeric Profilin

The present invention relates the production and purification of recombinant ZmPRO1 profilin, which yields multimeric forms. The protein was subsequently identified to be immunologically distinct through western immunoblot analysis and an ELISA that was developed to assess allergy patient IgE reactivity. An apparent tetrameric 60 kDa profilin was found in addition to higher multimeric orders (>97 kDa) that become amplified under non-reducing conditions. Although relatively high percentage acrylamide gels were used to visualize and study monomeric or lower multimeric orders of ZmPRO1 profilin, it appears that fastidious high profilin multimers persist; that is, despite exposure to heat and reducing agents to break up multimers, the larger multimeric profilin forms remained prevalent. The ability of plant profilin to form multimers was examined to determine optimal molecular forms for hyposensitization. Purified recombinant protein encoded by the ZmPRO1 cDNA was visualized by silver-stained SDS-PAGE separation (FIGS. 1, 2a). A predominant band at 14.8 kDa was identified in transformed E. coli, particularly after IPTG (isopropyl beta-D-thiogalaclopyranoside) to induce protein production. Other extraneous bands (>30 kDa) presumably included multimeric profilin and/or cellular proteins that remain with the more convenient PLP (poly-1-proline) affinity bead slurry. ZmPRO1 profilin separated by column chromatography yields a cleaner preparation that was also studied under both reduced and non-reduced conditions (FIG. 2a). A predominant band appeared as expected for monomeric ZMPRO1 plant profilin (approximately 14.8 kDa) but, consistent with reports for human profilin, higher molecular weight proteins such as higher multimeric profilin orders remained that were resistant to reducing agents. The band at approximately 60 kDa (FIG. 2a, +BME) suggested the formation of a tetramer resistant to reducing agents, in addition to the distinct aggregation of proteins near the top of the gel (>97 kDa). The larger proteins become more pronounced under non-reducing conditions (FIG. 2a, −BME) and are associated with a corresponding loss of monomeric profilin. The presence of stained proteins that remained in the stacking gel further supported the finding of natural protein (i.e. profilin) aggregation/multimerization. Any faint protein represented at 14.8 kDa in the absence of reducing agent became more evident on development of corresponding immunoblots with sensitive substrates (FIG. 2b). However, Western immunoblotting gave inconsistent positive identification for the higher molecular weight profilin multimers; presumably this was due to the different efficiencies in transfer of various protein sizes, alternations in net charge that may occur on protein-protein interactions (e.g. ionic bonds), difficulty for >90 kDa profilin multimers in migration from the stacking gel to the separating gel, or a lack of antibody recognition due to epitope masking when profilin aggregates/multimerization occurs. Indeed, differences in the observed Western immunoblot band intensities (monomer >tetramer >>higher orders) support these explanations. Collectively, the results suggest that immunologically distinct Zea mays profilin was produced, purified, and preferentially forms multimers.

### Example 2

### Evidence that Human Serum From Allergic Individuals Recgonizes ZmPRO1

An ELISA was developed to further immunologically identify the purified recombinant protein and to provide a means to study whether human serum from allergic individuals recognizes ZmPRO1 profilin immunologically. Six representative control wells are shown (FIG. 4), of which only those coated with the purified protein elicited a significant calorimetric response. In addition, rabbit antihuman profilin IgG did not recognize ZmPRO1 profilin, which is similar to the inability of rabbit anti-plant profilin IgG to recognize human profilin (Karakesisoglou et al., 1996). Thus, a method was established with a clear signal-to-noise ratio that was selective for ZmPRO1 profilin and further verified the production of immunologically distinct plant profilin.

Materials and Methods

Reagents

The cDNA encoding an isoform of profilin derived from the pollen of Zea mays (ZmPRO1) (Staiger et al., 1993) was provided in a transfection vector (pET23a; Novagen, Madison, Wis., USA) with an isopropyl beta-D-thiogalaclopyranoside (IPTG)-inducible promoter; polyclonal rabbit IgG that recognizes the protein product encoded by ZmPRO1 cDNA was also provided. Cyanogen bromide (CNBr)-activated sepharose 4B was purchased from Pharmacia (Piscataway, N.J., USA) and poly(L-proline) (PLP; 10000-30000 MW) was purchased from Sigma Chemical Co. (St. Louis, Mo., USA).

Horseradish peroxidase (HRP)-conjugated monoclonal antibodies (goat antirabbit IgG, goat antihuman IgE) and silver staining kits were purchased from Pierce Chemical Co. (Rockford, Ill., USA).

Patient Serum Samples

Human serum samples from routine blood draws were obtained from the University of Illinois College of Medicine at Rockford, Office of Family Practice (Rockford, Ill., USA) with appropriate patient consent; patient-declared allergies were annotated on each serum container. Serum isolated from whole blood by standard centrifugational methods was either stored at 4 C (and used within 1 week) or aliquoted and stored at −20 C. The samples were categorized into one of three groups: (i) no declared allergies; (ii) miscellaneous reactions (i.e. non-plant allergens, such as dust, adhesive tape, synthetic materials and the like); or (iii) classical Type I allergies to plant pollens.

Expression and Purification of ZmPRO1 Profilin

Pre-thawed competent BL21 (DE3) Escherichia coli cells (Novagen Inc., Madison, Wis., USA) were transformed with ZmPRO1/pET-23a by a modified protocol from the manufacturer and essentially as described for plants (Vrtala, et al, 1996; Susani, 1995; Karakesisoglou, 1996) and human profilins (Gieselmann et al., 1995). The DNA content and quality from lysates of various transformed E. coli clones were analyzed by standard spectrophotometric measurement (i.e. 260 nm, concentration; 260 nm/280 nm, relative nucleotide purity versus protein) and argarose (0.7%) gel electrophoresis. The E. coli clones expressing the highest concentrations of ZmPROl cDNA were selected for profilin production.

Transformed E. coli initially grown in 10 mL L-broth (in g/L: 10 tryptone, 5 bacto yeast extract, 10 NaCl+0.15 ampicillin) at 37° C. for 10 h were brought to a final 1 L volume of L-broth and incubated for an additional 2 h (37 C, gentle mixing at 100 r.p.m.) prior to the addition of either IPTG (0.4 mmol/L final concentration) or vehicle for an additional 6 h incubation. The cultures were centrifuged (1000 g for 30 min at 22° C.) to yield pellets that were resuspended in 5 volumes of ice-cold lysis buffer (0.01% Triton X-100, 2 μmol/L leupeptin, 1 μmol/L aprotinin, 0.2 μmol/L pepstatin, 5 mmol/L This-HCl, pH 7.2) and sonicated (continuous output control setting 2×10 s, Sonifier cell disruptor, Branson Sonic Power Ca., Danbury, Conn., USA). The lysates were centrifuged (12 000 g for 30 min at 4° C.) and supernatant poured onto poly(L-proline)-sepharose 4B (i.e. PLP bead) affinity column, as described previously (Babich et al., 1996; Janmey, 1991). Briefly, a step-wise elution gradient with urea was used to collect and purify profilin for overnight dialysis (at 4° C.) against 2 mmol/L N-2-hydroxyethylpiperazine-N′-2-ethanesulfonic acid (HEPES), pH 7.2/0.1 mmol/L CaCl₂ and concentration by centrifugation (centriplus-3, Amicon Inc., Beverly, Mass., USA) to a final concentration of approximately 1 mg/mL, which was stored at −20 C. In some cases, ZmPRO1 profilin was isolated by a co-incubation of E coli lyrate: PLP bead slurry (1:4 vol; 4-16 h at 4°0 C., gentle shaking), followed by centrifugation to pellet and wash the profilin-PLP bead complexes (3 times with 100 mmol/L NaCl, 100 mmol/L glycine, 0.01 mmol/L DTT, 10 mmol/L Tris base, pH 7.8). The final pellet was suspended and boiled in sample buffer, with or without β-mercaptoethanol (BME).

Proteins isolated by either PLP bead slurry (e.g. FIG. 1; initial purification and validation that profilin was made in E. coli) or column chromatography were analyzed by standard silver-stained sodium dodecyl sulfate-polyacrylamide gel electrophoresis (SDS-PAGE; 15% acrylamide) techniques. Profilin was further characterized by western immunoblotting, as previously described (Babich et al., 1996). The immunoblot was developed by incubation with rabbit anti-ZmPRO1 primary antibody (1:1000) and goat antirabbit secondary antibody conjugated with horseradish peroxidase (1:500). Proteins were visualized with either a flourescent substrate or enhanced metal substrate (Super Signal or metal diarninobenzidine tetrahydrochloride (DAB), Pierce Chemical Co., Rockford, Ill., USA).

### Anti-Profilin Antibody Development

Profilin antibodies were made against either recombinant or native profilin as described (13abich, et al., 1996; Staiger et al., 1993). After affinity column purification and SDS-PAGE electroelution, the profilin was conjugated with adjuvant (RIBI Immunochemical Research, Inc., Hamilton, Mont.) and injected into ten discrete locations (per RIBI protocol) into New Zealand White rabbits. Rabbit serum anti-profilin IgG was purified by thiophilic adsorption chromatography (Pierce Chemical Co., Rockford, Ill.) to yield an average peak fraction concentration of 4.5 mg IgG/ml. Anti-profilin IgG antibodies were screened on Western immunoblots containing antigen. Enzyme-linked immunosorbant assay for human IgE-ZmPRO1 profilin detection Purified ZmPRO1 product (50 ng/well) or a control vehicle [tris-buffered saline (TBS), pH 7.4] was added to designated wells of a 96-well immunoassay plate (Immulon-2, Dynatech Laboratories Inc., Chantilly, Va., USA) that was stored overnight at 4 C. The general sequence to develop the appropriate wells for the ELISA was as follows: (i) block non-specific sites (4% non-fat powdered milk, 0.1% bovine serum albumin (BSA), 0.02% NaN₃ in TBS, 20% SuperBlock from Peirce Chemical Co. for 2 h at 4 C); (ii) 1× TBS wash and incubate with either serum samples or a control vehicle (TBS or heat-inactivated fetal calf serum; overnight at 4° C.); (iii) discard samples and add either TBS to sample wells or primary rabbit IgG antiplant profilin (1:1000 dilution in TBS, 0.01% Tween-20, 0.01% BSA for 1.5h) into control wells to ensure ZmPRO1 profilin coating; and (iv) 1× TBS wash of all wells, add appropriate secondary antibodies (either goat antihuman IgE-HRP in wells that contained serum or goat antirabbit IgG-HRP in control wells; 1:500 dilutions for 2 h at 4° C.). The plate was extensively washed then developed using 2,2′-azino-bis (3-ethylbenzthiazoline-6-sulfonic acid) as a colorimetric method substrate (although other established HRP substrates were also successful in preliminary work) and optical densities were measured with a microtiter platereader (λ₅₇₀ₙₘ). Serial dilution assay of profilin standards indicated that the assay was linear between 0.1 and 100 ng additions of profilin per well. Serum samples were considered reactive with profilin (i.e. positive) if there was at least one standard deviation difference between the optical densities obtained from the wells containing profilin versus those without profilin. Serum identified as positive gave a linear increase in signal intensity of concentrations between 10 and 100%.

Dot-Filtration Immunoblot

In some instances, a dot filtration immunoblot assay (Bio Rad, Hercules, Calif., USA) using supported nitrocellulose (0.2 μm pore size) was a necessary alternative to the ELISA to determine the allergenic potential of ZmPRO1 monomers versus multimers. Profilin was either placed under reducing (4.5% BME at 95 C for 3 min) or non-reducing conditions (to favor monomeric or multimeric conditions, respectively) and subsequently allowed to adhere to the dot immunoblot (50 ng/well for 2 h) prior to vacuum filtration to remove the medium from the membrane. The BME was then removed by thorough washing with TBS and the dot immunoblot was developed with antibodies similar to the ELISA method, but with an enhanced metal DAB as the HRP substrate. Quantitative values for the intensity of immunorecognition (i.e. darkness) were obtained by computer scanning the dotfiltration immunoblot and using Adobe Photoshop (Adobe systems, San Jose, Calif., USA) software program (under histogram, black channel). The average brightness value was obtained from the fixed number of pixels (486) that covered each dot and the corresponding darkness value was calculated by 100× the inverse of the brightness (i.e. increased relative value represents increased darkness or immunoreactivity). Comparisons between the means of different treatments were made by Student's t-test (Sokal and Rohlf, 1981).

Methods of Administration to Hyposensitize or Desensitize a Mammal

The present invention covers the use of profilin polypeptide allergens, e.g. a fragment of multimer profilin, to hyposensitized or desensitize a mammal. Such polypeptides can be administered to a mammal either alone or in combination with pharmaceutically acceptable carriers or diluents, in accordance with standard pharmaceutical practice.

The method of hyposensitization involves, the successive parenteral, oral, nasal, inhalant or rectal administration of incremental doses of profilin. The term parenteral as used herein includes subcutaneous, intravenous or intramuscular injections.

A range from approximately 1 picogram to 10 milligrams per application dose can be used as an “effective dose.” However, the amount and number of administrations sufficient to produce clinical effectiveness as measured by reduced IgE-related symptoms; diagnostic use or dosage is the amount which is sufficient to produce, or include in, a method to measure a reaction. The diluents and carriers are chosen by those skilled in the art according to commonly accepted clinical procedures.

### DOCUMENTS CITED

Astwood J D, et al., Nat. Biotech. 1996; 14; 1269-73.

Baalout S, Eur. J. Clin. chem. Clin. Biochem. 1996; 34: 575-7.

Babich M., et al., Biochem. Biophys. Res. Commun. 1996; 218: 125-31.

Carlsson L, et al., J. Mol. Biol. 1976; 105: 353-66.

Gieselmann, et al., Eur. J. Biochem. 1995; 229: 621-8.

Goldschmidt-Clermont P J, Janmey P A, Cell 1991; 66; 419-21.

Heiss S, et al., Allergy Clin. Immunol. 1996; 9B; 938-47.

Honore, B, et al., FEBS 1993; 330, 151-5.

Janrney P A, Meth. Enzymol. 1991; 196: 92-9.

Karakesisoglou, et al., Cell Mot. Cyloskel. 1996; 34; 36-47.

Kwiatkowski, D J and Bruns, G A P, J. Biol. Chem. 1988; 263, 5910-5.

Lassing I, Lindberg U, Nature 1985; 314: 472-4.

Metzler et al., Protein Science 1995; 4(3):450-9.

Mittermann I, et al., Sexual Plant Reprod. 1998; 11: 183-91.

Pauli G., et al., Allergy Clin. Immunol. 1996; 97: 1100-9.

Psaradellis, T. et al. Allergology Int. 2000; 49:27-35.

Sambrook J, Russell D W, Molecular Cloning: A Laboratory Manual, 3^{rd} ed., 2001.

Sohn R H, Goldschmidt-Clermont P J, Bioessays 1994; 16: 165.72.

Sokal R R, Rohlf F J, Biometry, 2^{nd} edn. San Francisco: WH Freeman; 1981.

Staiger C J, et al., Plant J. 1993; 4: 631-41.

Susani M., Biochem. Biophys. Res. Commun. 1995; 215: 250-63.

Theriot J A, Mitchison T J, Cell 1993; 75; 835.8.

Valenta R., et al., Science 1 1991; 253: 557-60.

Valenta R., Kraft D, Curr. Opin. Immunol. 1995; 7: 751-6.

Valenta R, et al., J. Biol. Chem. 1993; 268: 22 777-81.

Valenta R., et al., J. Exp. Med. 1992a; 175: 377-85.

Valenta R., et al., Int. Arch. Allergy Immunol. 1992b; 99: 271-3.

Vallier P, et al., Clin. Exp. Allergy 1995; 25: 332-9.

Vrtala S., et al., Biochem. Biophys. Res. Commun. 1996a; 226: 42-50.

Vrtala S. et al., J. Allergy Clin. Immunol. 1996b; 98: 913-21.

Weldemann P, et al., J. Biol. Chem. 1996; 271: 29 915-21.

Wellhausen A, et al., Zeitschrift Ernahrungswissenschaft 1996; 35: 248-355.

U.S. Pat. No. 5,583,046.

U.S. Pat. No. 5,648,242.

We claim:

1. A method to hyposensitize a mammal, said method comprising:
(a) obtaining an immunogenic composition comprising multimeric profilin; and
(b) administering an effective dose of the composition successively in incremental doses until the mammal is hyposensitized.

2. The method of claim 1, wherein multimeric profilin is selected from a group consisting of naturally occurring, synthetic, or recombinantly made profilin

3. The method of claim 2, wherein the profilin occurs as complexes of homomultimers.

4. The method of claim 1, wherein the multimeric profilin comprises synthetic peptide fragments of profilin.

5. The method of claim 3, wherein the multimeric profilin comprises synthetic peptide fragments that have novel sequences that arise from profilin multimerization

6. The method of claim 1, wherein the multimeric profilin comprises peptide fragments made by recombinant DNA technology.

7. The method of claim 1, wherein the multimeric profilin comprises monomers selected from the group consisting of celery (Api g4, GENE BANK ACCESSION NO. QPXF37), peanut (Ara h5, GENE BANK ACCESSION NO. Q9SQ19), birch tree pollen (Bet v2, GENE BANK ACCESSION NO. P25816), Bermuda grass (Cyn d12, GENE BANK ACCESSION NO. 004725), soybean (Gly m3, GENE BANK ACCESSION NO. 065809, 065810), sunflower (Hel A2, GENE BANK ACCESSION NO. 081980), latex (Hev b8, GENE BANK ACCESSION NO. CAB51914, 065812, Q9STB6, Q9M7NO, Q9M7M9, Q9M7M8, Q9LE18), Mercurialis annua (Mer al, GENE BANK ACCESSION NO. 049894), olive tree pollen (Ole e2, GENE BANK ACCESSION NO. P19963, 0024170m 024171), timothy grass (Phl p11, GENE BANK ACCESSION NO. P35079, 024650, 024282), sweet cherry (Pru av4, GENE BANK ACCESSION NO. Q9XF39), pear (Pyr c4, Q9XF27), corn pollen (Zea Pro I, GENE BANK ACCESSION NO.B35081; Zea Pro II, GENE BANK ACCESSION NO. P35080; ZMPro III, GENE BANK ACCESSION NO. P35083; ZmProIV, GENE BANK ACCESSION NO. 022655; ZmProV, GENE BANK ACCESSION NO. Q9FR39), human (profilin I, GENE BANK ACCESSION NO. P07737; Profilin II isoform 1, GENE BANK ACCESSION NO. NP_{—}444252; and Profilin II isoform GENE BANK ACCESSION NO.NP_{—}002619, or combinations thereof.

8. The method of claim 1, wherein the immunogenic composition comprises pharmaceutically acceptable carriers or diluents.

9. The method of claim 1, wherein administration route is selected from the group consisting of parenteral, oral, nasal, inhalant or rectal routes.

10. A synthetic peptide that is characterized as:
(a) including from about 7 to 21 amino acids sequentially linked and selected from the monomeric profilin of Table 3;
(b) the amino acids include at least one proline; and
(c) the amino acids include at least one acidic amino acid; and

11. The synthetic peptide of claim 9, wherein the sequence of the amino acids is the same novel sequence that is found by continuing the sequence from one profilin molecule to the adjoining profilin molecule as shown in FIG. 5 in the binding regions exposed by aggregation.

12. A profilin multimer comprising an aggregation of synthetic peptides of claim 10.

13. The multimeric form of the plant profilin of claim 11, further defined as heteromultimeric.

14. The multimeric form of plant profilin of claim 11 further defined as synthetic.

15. Purified multimeric forms of plant profilin having the following characteristics:
(a) comprising monomeric plant profilin each monomer having an amino acid sequence that each contain at least one cystei or fragments thereof; and
(b) having an estimated molecular weight of at least 24 kDa.

16. A composition wherein the multimeric profilin is selected from the group of monomeric profilin consisting of celery (Api g4, GENE BANK ACCESSION NO. Q9XF37), peanut (Ara h5, GENE BANK ACCESSION NO. Q9SQ19), birch tree pollen (Bet v2, GENE BANK ACCESSION NO. P25816), Bermuda grass (Cyn d12, GENE BANK ACCESSION NO. 004725), soybean (Gly m3, GENE BANK ACCESSION NO. 065809, 065810), sunflower (Hel A2, GENE BANK ACCESSION NO. 081982), latex (Hev b8, GENE BANK ACCESSION NO. CAB51914, 065812, Q9STB6, Q9M7NO, Q9M7M9, Q9M7M8, Q9LE18), Mercurialis annua (Mer al, GENE BANK ACCESSION NO. 049894), olive tree pollen (Ole e2, GENE BANK ACCESSION NO. P19963, 0024170, 024171), timothy grass (Phl p11, GENE BANK ACCESSION NO. P35079, 024650, 024282), sweet cherry (Pru av4, GENE BANK ACCESSION NO. Q9XF39), pear (Pyr c4, Q9XF27), corn (Zm PROI, GENE BANK ACCESSION NO. P35081; Zm Pro II, GENE BANK ACCESSION NO. P35082; ZmPro III, GENE BANK ACCESSION NO. P35083; ZmProIV, GENE BANK ACCESSION NO. 022655; ZmProV, GENE BANK ACCESSION NO. Q9FR39), human (profilin I, GENE BANK ACCESSION NO. P07737; Profilin II isoform a, GENE BANK ACCESSION NO. NP_{—}444252; and Profilin II isoform b, GENE BANK ACCESSION NO.NP 002619, or composition thereof.

17. A diagnostic test for allergies, said test comprising:
(a) obtaining a pharmaceutical composition of multimeric profilin or functionally equivalent fragments thereof;
(b) administering the composition to a subject; and
(c) determining a reaction from which alllergenicity is inferred.

18. Use of multimeric profilin or a functional equivalent to hyposensitize a mammal.

19. An antibody directed against a synthetic peptide of claim 10.

20. An antibody directed against the multimeric profilin of claim 12.

21. An antibody against the multimeric profilin of claim 15.
